(19) United States
(12) Patent Application Publication
(10) Pub. No.: US 2010/0004294 A1
(43) Pub. Date: Jan. 7, 2010

(54) Stable Lozenge Compositions Providing Rapid Release of Nicotine

(75) Inventors: Anders Axelsson, Bjarred (SE); Arne Kristensen, Helsingborg (SE); Henri Hansson, Helsingborg (SE)

Correspondence Address: EDWARDS ANGELL PALMER & DODGE LLP, P.O. BOX 55874, BOSTON, MA, 02205, US

(73) Assignee: NicoNovum AB, Helsingborg (SE)

(21) Appl. No.: 12/226,273

(22) PCT Filed: Sep. 16, 2007
(86) PCT No.: PCT/EP2007/002345

§ 371 Date: Apr. 13, 2009

Related U.S. Application Data
(60) Provisional application No. 60/782,903, filed on Mar. 16, 2006.

(30) Foreign Application Priority Data
Mar. 16, 2006 (DK) PA 2006 00375

(51) Int. Cl. A61K 31/465; A61P 25/34

(52) U.S. Cl. 514/343

(57) ABSTRACT

Compositions comprising nicotine, which compositions provide a rapid release of nicotine. Nicotine is present in the form of a nicotine-cellulose combination. The compositions are designed for administration to the oral cavity where the nicotine is rapidly released from the composition and available for absorption through the oral mucosa. The compositions are lozenges and have excellent storage stability.

### FIELD OF THE INVENTION

The present invention relates to compositions comprising nicotine, which compositions provide a rapid release of nicotine. Nicotine is present in the form of a nicotine-cellulose combination. The compositions are designed for administration to the oral cavity where the nicotine is rapidly released from the composition and available for absorption through the oral mucosa. The compositions are lozenges and have an excellent storage stability.

### BACKGROUND OF THE INVENTION

Smoking behavior is associated with serious health risks not only to the smoker, but also to the people around him exposed to passive smoke. To quit smoking has therefore been the expert's advice for many years. However, the smoker is addicted to nicotine, which makes quitting quite difficult for most smokers. Other ways of nicotine administration have been employed in the efforts to help smokers quit their unhealthy habit. Several products employing oral or transdermal administration of nicotine are currently available for smokers wanting to quit smoking, such as chewing gums, inhalators, patches or mouth sprays.

As the tobacco itself contains several other toxic compounds other than nicotine, nicotine substitution products are also relevant for individuals who consume their tobacco in other ways than by smoking. Mainly in Scandinavia, particularly in Sweden, tobacco is consumed as chewing tobacco or snuff. The use of nicotine substitution products will spare consumers of chewing tobacco or snuff as well as smokers from the carcinogenic risks derived from tobacco.

In spite of the availability of several nicotine substitution products such as those mentioned above, many individuals addicted to nicotine still find it difficult to quit their consumption of tobacco. The explanation for this is probably a combination of multiple factors, of which two of them relate to the attained concentration of nicotine in the bloodstream and more importantly, the rate by which nicotine reaches the bloodstream and thereby provides the user with the desired effect.

The rate by which nicotine reaches the bloodstream can be limited by the in vitro rate by which nicotine is released from the nicotine substitution product. Accordingly, there is a need for pharmaceutical compositions comprising nicotine with a rapid release of nicotine, e.g. a rapid in vitro and/or in vivo release. Furthermore, rapid release of nicotine minimizes the total content of nicotine necessary in the compositions, which is a benefit in terms of the consumer's total intake of this potentially toxic compound and in terms of manufacturing economy.

Longenze compositions comprising nicotine have been described before, but to the best of the inventor's knowledge it has not been possible to provide a fast releasing nicotine-containing lozenge composition that without any firm sealing of the package has sufficient storage stability. The stability problems are mainly related to the volatility of the nicotine (which as a free base is in liquid form).

### SUMMARY OF THE INVENTION

The present invention addresses the above-mentioned problems by providing a composition that provides a rapid release of nicotine and thus, provides a rapid increase in the plasma concentration of nicotine upon in vivo use. The composition is in the form of a tablet for buccal absorption (i.e. in the form of a lozenge) and it has a shelf life of 24 months or more. The composition may be used as a pharmaceutical composition and/or as a tobacco substitute composition.

Thus, the present invention relates to the use of a nicotine-cellulose combination and one or more pharmaceutically acceptable excipients for the preparation of lozenge composition for achievement of a fast onset of action of nicotine after application of the composition to the oral cavity of a subject.

The inventors have found that in order to achieve a rapid initial release of nicotine, the dissolution time in vivo should be 15 minutes or faster. The in vivo dissolution time is determined as the time it takes from application of the lozenge and it is completely disintegrated.

Moreover, the present invention provides a lozenge composition that has a suitable storage stability and which do not need to be Barex sealed in order to avoid any disappearance of nicotine from the composition. As shown in the examples herein a storage stability of at least 2 years at room temperature is obtained.

In the present context the term “nicotine-cellulose combination” is intended to denote a solid material composed of a cellulose which has sorbed (adsorbed and/or absorbed) a well-defined amount of nicotine (either as free base or as a pharmaceutically acceptable salt, complex or solvate) e.g. in and/or onto voids or pores within the cellulose. The terms “nicotine-cellulose adduct” and “nicotine-cellulose carrier complex” as used herein are intended to have the same meaning as the term “nicotine-cellulose combination”. As used herein cellulose is an example of a carrier.

A composition of the invention has a fast initial release of nicotine, thus, the composition—when subjected to an in vitro release test—within the first 2 minutes after start of the test releases nicotine with a release rate corresponding to 10% w/w or more of the total content in the composition per minute.

A fast onset of the nicotine effect is very important in order to be an acceptable product for the consumer. Accordingly, for a lozenge composition of the invention, the onset takes place within 3 minutes such as, e.g., within 2.5 minutes or within 2 minutes after application of the composition to the oral cavity of the subject.

Accordingly, in another aspect, the invention relates to a composition in solid or semi-solid dosage form, notably a lozenge composition, comprising nicotine, or a pharmaceutically acceptable salt, solvate, complex, adduct, or derivative thereof, and one or more pharmaceutically acceptable excipients, wherein—when subjected to an in vitro dissolution test as described herein—within the first 2 minutes after start of the test releases nicotine with a release rate corresponding to 7.5% w/w or more of the total content in the composition per minute.

As it appears from the examples herein the present inventors have found that compositions in the form of a lozenge are especially suitable to achieve a fast release and a subsequent fast appearance of nicotine in the plasma upon in vivo use. Accordingly, in specific embodiments the invention relates to

i) a lozenge comprising nicotine, or a pharmaceutically acceptable salt, solvate, complex, adduct, or derivative thereof, and one or more pharmaceutically acceptable excipients, wherein—when subjected to an in vitro dissolution test as described herein—within the first 2 minutes after start of the test releases nicotine with a release rate corresponding to 7.5% w/w or more of the total content in the composition per minute; and

Furthermore, the present invention provides methods for preparation of such compositions, comprising mixing nicotine, or a pharmaceutically acceptable salt or derivative thereof, and one or more pharmaceutical acceptable excipients and forming it into a suitable solid dosage form.

The present invention also relates to the use of compositions according to the invention, for treatment of nicotine addiction and/or nicotine withdrawal symptoms.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### DETAILED DESCRIPTION OF THE INVENTION

In keeping with long-standing patent law convention, the words “a” and “an” when used in the present specification in concert with the word comprising, including the claims, denote “one or more.” Some embodiments of the invention may consist of or consist essentially of one or more elements, method steps, and/or methods of the invention. It is contemplated that any method or composition described herein can be implemented with respect to any other method or composition described herein.

As mentioned above, the present invention relates to nicotine-containing compositions that release nicotine very fast in order to achieve a very fast rise in plasma concentration upon administration, especially by the oral mucosa. In particular, the invention relates to compositions in a form that is suitable for delivering nicotine to the oral mucosa such as lozenges.

In a first aspect, the invention relates to a lozenge composition in solid or semi-solid dosage form comprising nicotine, or a pharmaceutically acceptable salt, solvate, complex, adduct, or derivative thereof, and one or more pharmaceutically acceptable excipients, wherein—when subjected to an in vitro dissolution test as described herein—within the first 2 minutes after start of the test releases nicotine with a release rate corresponding to 7.5% w/w or more of the total content in the composition per minute. As demonstrated in the examples herein, such a fast release is not obtained by marketed compositions in the form of chewing gum such as Nicorette®. To this end, the present inventors have found that especially a lozenge composition in the form of a tablet offers advantages over the Nicorette® chewing gum compositions and, furthermore, the use of a nicotine-containing compound in a specific form may also be advantageous in order to obtain as fast a release as possible.

More specifically, the above-mentioned release rate within the first 2 minutes after start of the test is 10% w/w or more such as, e.g., 11% w/w or more, 12% w/w or more, 13% w/w or more, 14% w/w or more or 15% w/w or more of the total content in the composition per minute.

In a specific embodiment, the composition according to the invention further comprises microcrystalline cellulose (“mcc”). Certain specific embodiments may also utilize other forms of carriers, in addition to or including mcc, such as but not limited to fibrous material or carbohydrates including cellulose (including hemicellulose, celluloses with different crystallinities and structures (e.g., varying structures including solid fibers, and addition or including fibers or the like in various structures such as web-like structures and/or other structures), including naturally occurring celluloses including Cladophora sp. Algae cellulose or the like), dextran, agarose, agar, pectin, alginate, xanthan, chitosan, starch (including potato starch, shoti starch) etc. or mixtures thereof.

Nicotine may be present in any suitable form such as, e.g. in the form of the free base form of nicotine or in the form of a suitable salt or complex thereof. Moreover, the nicotine may be present in the form of a carrier complex or a carrier adduct, wherein nicotine is present together with a carrier compound. In a specific embodiment, the carrier compound is a particulate material comprising internal voids throughout the material and the voids at least partially comprises said nicotine. While not intended to be bound by theory, it is believed as of the time of this patent application that nicotine may interact the carrier (for example, mcc or other suitable carrier including other cellulose carriers) by absorbing into and/or adsorbing onto the carrier. Such interaction is completely or nearly completely reversible.

A particular suitable material having internal voids is a cellulose such as, e.g., a microcrystalline cellulose. Specific examples of a suitable microcrystalline cellulose is microcrystalline cellulose selected from the group consisting of AVICEL® grades PH-100, PH-102, PH-103, PH-105, PH-112, PH-113, PH-200, PH-300, PH-302, VIVACEL® grades 101, 102, 12, 20 and EMOCEL® grades 50M and 90M, and the like, and mixtures thereof.

The microcrystalline cellulose may be a synthetic or semi-synthetic cellulose, or it may be derived from natural celluloses.

Suitable carriers may also be those disclosed in WO 2004/064811, which is hereby included by reference.

More specifically, it is contemplated that a relatively high surface area may be of importance for a carrier that is suitable for use. Accordingly, the specific surface area of suitable carriers is normally at least 0.7 m²/g such as, e.g., 1 m²/g. In certain uses, the specific surface area may range between about 0.7 m²/g and at least about 100 m²/g and/or may be anything within this range and/or may be any mixture of sizes within this range. For example, in certain embodiments, the surface area may be about 0.7 m²/g, about 1 m²/g, about 1.5 m²/g, about 2.0 m²/g, about 3.0 m²/g, about 5 m²/g, about 7 m²/g, about 10 m²/g, about 15 m²/g, about 20 m²/g, about 25 m²/g, about 35 m²/g, about 45 m²/g, about 50 m²/g, about 75 m²/g, about 100 m²/g and above about 100 m²/g, or combinations thereof. Such carriers having such suitable surface areas may include, but are not limited to, mcc, fibrous material or carbohydrates including cellulose (including hemicellulose, celluloses with different crystallinities and structures (e.g., varying structures including solid fibers, and addition or including fibers or the like in various structures such as web-like structures and/or other structures), including naturally occurring celluloses including Cladophora sp. Algae cellulose or the like), dextran, agarose, agar, pectin, alginate, xanthan, chitosan, starch (including potato starch, shoti starch) etc. and/or mixtures thereof.

Normally, the mean size range of the carrier compound is from about 15 to about 250 μm.

The present inventors have found that a small particle size of the cellulose employed is advantageous in order to avoid a rough or gritty mouthfeell after application. Accordingly, a mean particle size of about 20 μm is suitable and an example of such a material is Avicel PH-105.

More specifically, in an embodiment of the invention, nicotine is present as a nicotine-cellulose combination in which said nicotine is at least partly sorbed on microcrystalline cellulose and/or is at least partially absorbed into the carrier and/or is at least partially adsorbed onto the carrier (e.g., mcc), or mixtures thereof. Such interaction is completely or nearly completely reversible.

Hence, in certain specific embodiments nicotine is sorbed on microcrystalline cellulose, absorbed into the mcc and/or adsorbed onto the mcc, and/or combinations thereof.

In embodiments of the present invention, the carrier (e.g., but not limited to mcc and/or other naturally-occurring cellulose) is at least partially porous. This porosity may be due, for example but not limited to, the structure of the carrier, for example, branched, fiberous, or weblike structures may have pores. Ranges of pore sizes include but are not limited to pore volumes of about 0.01 cm³/g and include, but are not necessarily limited to pore volume ranges of from about 0.003 cm³/g or less to about 0.025 cm³/g, to about or greater than 0.60 cm³/g.

In general, the nicotine-cellulose combination is present in a composition of the invention in a concentration of at least about 2% w/w such as in a range from about 2% w/w to about 98% w/w, from about 2% to about 96% w/w, from about 2% w/w to about 95% w/w, from about 3% w/w to about 90% w/w, from about 4% w/w to about 85% w/w, from about 5% w/w to about 80% w/w, from about 5% w/w to about 75% w/w, from about 5% w/w to about 70% w/w, or from about 7.5% w/w to about 65% w/w.

In certain embodiments, the amount of nicotine sorbed, for example absorbed into and/or adsorbed onto to carrier can be up to 50% or more of the total weight of the composition. Ranges of the amount of nicotine sorbed onto the carrier in the present invention range for less than about 1% of the total weight of the composition to more than about 50% of the composition, including all amounts within this range. While applicants do not intend the invention to be bound by theory, it is believed at the time of preparing this application that the maximum amount of nicotine that can be sorbed onto and/or into the carrier, thereby affecting the amount, for example the percent nicotine by weight of the total composition (e.g., the maximum percentage) is affected by properties of the carrier, including but not limited to the structure of the carrier, the porosity of the carrier, and the surface area of the carrier.

In specific embodiments, the concentration of the nicotine carrier complex or nicotine carrier adduct in a composition of the invention is present in a concentration such as, e.g., from about 2% w/w (of the total composition) to about 20% w/w, from about 4% w/w to about 19% w/w, from about 5% w/w to about 18% w/w, from about 6% w/w to about 17% w/w, from about 7% w/w to about 16% w/w or from about 8% w/w to about 15% w/w. In particular this is the case in those situations where the dose required of nicotine is relatively small such as, e.g., in up to a 10 mg range.

In an alternative embodiment, the carrier compound is capable of forming a complex with nicotine such as, e.g., in the case that the carrier compound is an ion-exchange compound including polacrilex.

Concentrations and Amounts of Nicotine

As mentioned above, nicotine may be present in any suitable form. Normally, nicotine is selected from the group consisting of nicotine base, nicotine hydrochloride, nicotine dihydrochloride, nicotine monotartrate, nicotine bitartrate, nicotine sulfate, nicotine zinc chloride such as nicotine zinc chloride monohydrate and nicotine salicylate. In a preferred aspect, nicotine is in its free base form, which easily can be sorbed on a cellulose to form a microcrystalline cellulose-nicotine carrier complex or carrier adduct.

The nicotine is normally present in the composition in a concentration from about 0.1% w/w to about 90% w/w such as, e.g., from about 0.1 to about 80% w/w, from about 0.1 to about 70% w/w, from about 0.1 to about 60% w/w, from about 0.1 to about 50% w/w, from about 40% w/w, from about 0.1 to about 30% w/w, from about 0.1 to about 20% w/w, from about 0.1 to about 10% w/w, normally from about 0.1 to about 5% w/w.

Normally, the nicotine compound (calculated as the free base) is present in a concentration of at least about 0.1% w/w such as in a range from about 0.1% w/w to about 50% w/w such as, e.g., from about 0.5% w/w to about 45% w/w, from about 1.0% w/w to about 40% w/v, from about 1.5% w/w to about 35% w/w, from about 2% w/w to about 30% w/w, from about 2.5% w/w to about 25% w/w, from about 2.5% w/w to about 20% w/w, from about 3% w/w to about 15% w/w.

Especially in compositions containing a relatively small amount of nicotine (e.g. chewing gums), the concentration of the nicotine compound (calculated as the free base) is normally in a range from about 0.1% wtw to about 15% w/w such as, e.g., from about 0.1% w/w to about 14% w/w, from about 0.1% w/w to about 13% w/w, from about 0.1% w/w to about 12% w/w, from about 0.1% w/w to about 11% w/w, from about 0.1% w/w to about 10% w/w as calculated as free nicotine base.

As mentioned above, the nicotine is present in the form of a nicotine-cellulose combination. In general, this combination is present in a concentration of from about 5% to about 100% such as, e.g., from about 10 to about 100%, from about 5% to about 50% or, alternatively, from about 45% to about 100%. The choice of suitable concentration depends on the load of nicotine in the nicotine-cellulose combination and the dosage of nicotine. If the load is relatively high, then the concentration of the combination may be lower than if the load is relatively low and vice versa. In a specific embodiment using e.g. Avicel® or a similar cellulose quality a concentration of the combination is generally from about 80% w/w to about 98% w/w, such as, e.g., from about 85% w/w to about 98% w/w, from about 90% w/w to about 98% w/w, from about 92% w/w to about 98% w/w, from about 93% w/w to about 97% w/w or from about 94% w/w to about 96% w/w.

The concentration of nicotine (or the pharmaceutically acceptable salt, complex or solvate thereof in the combination is at the most 70% w/w such as, e.g., at the most 60% w/w, at the most 50% w/w, at the most 45% w/w. The content of nicotine must not be so high that the combination (which is in powder form) “sweats”, so that nicotine desorbs, evaporates or otherwise disappears from the combination. Accordingly, the load of nicotine in the combination is dependent on the particular cellulose employed. If the surface area of the cellulose material is relatively high, then a larger amount of nicotine can be contained therein in a stable manner during a suitable period of time, whereas a cellulose having a smaller surface area normally is indicative for a lower capacity to load nicotine in a suitable manner with respect to stability.

For most cellulose qualities, the concentration of nicotine in the nicotine-cellulose combination is at the most about 45% w/w, such as, e.g., at the most about 40% w/w, at the most about 35% w/w, at the most about 30%.w/w, at the most about 25% w/w, at the most about 20% w/w, at the most about 15% w/w, at the most about 12.5% w/w, at the most about 10% w/w, at the most about 9.5% w/w, at the most about 9% w/w, at the most about 8.5% w/w or at the most about 8% w/w, and the concentration being calculated as the nicotine base.

The amount of the nicotine compound (calculated as the free base) in a composition of the inventions is generally from about 0.5 mg to about 10 mg such as, e.g., from about 1 mg to about 8 mg, from about 1.5 mg to about 7.5 mg, from about 2 mg to about 5 mg, from about 2.5 mg to about 5 mg, from about 3 to about 10 mg, from about 3 to about 7.5 mg or from about 3 mg to about 5 mg such as, e.g., about 1.5 mg, about 2 mg, about 2.5 mg, about 3 mg, about 3.5 mg, about 4 mg, about 5 mg or about 6 mg, as calculated as free nicotine base. In particular a dosage of 2 mg, 3 mg, 4 mg and 6 mg is of commercial interest. The tablet weight is in a range of from about 50 mg to about 700 mg such as 600-650 mg.

Buffering Agents

A composition according to the invention may also contain one or more buffering agents. It is generally known that a slightly alkaline reaction (between 7 and 8) in the oral cavity enhances the absorption of nicotine. Accordingly, it may be and advantage to incorporate a buffer substance in the composition such that a slightly alkaline reaction is provided. Especially compositions for release of the nicotine in the oral cavity can advantageously contain a buffer substance, i.e. compositions like chewing gums, lozenges and snuff compositions.

Suitable buffering agents are typically those selected from the group consisting of acetates, glycinates, phosphates, glycerophosphates, citrates such as citrates of alkaline metals, carbonates, hydrogen carbonates, and borates, and mixtures thereof.

If present the one or more buffering agents are present in a concentration from about 0.5% w/w to about 5% w/w, such as, e.g., from about 0.75% w/w to about 4%, wIw, from about 0.75% w/w to about 3%, w/w or from about 1% w/w to about 2%, w/w.

Sweeteners

In order to increase the sensoric properties of the composition according to the invention one or more sweeteners may be added, such as sugar alcohols including xylitol, sorbitol and/or isomalt, or artificial sweeteners such as e.g. aspartame, acesulfame or saccharin.

The concentration of the one or more sweeteners, if present, is normally at least about 0.05% such as, e.g. from about 0.075% w/w to about 5% w/w or from about 5% to about 35% w/w, such as, e.g., from about 10% w/w to about 35% w/w, from about 15% w/w to about 35% w/w or from about 20% w/w to about 30% w/w.

Anti-Oxidants

It is well-known that nicotine is subject to oxidation and accordingly, it may be advantageous to incorporate one or more anti-oxidants, such as, e.g., ascorbyl palmitate and/or sodium ascorbate, in a composition according to the invention.

The one or more anti-oxidants may be present in a concentration of from about 0.05% w/w to about 0.3% w/w, such as, e.g., from about 0.1% w/w to about 0.25% w/w or from about 0.15% w/w to about 0.2% w/w.

Flavouring Agents

In order to improve the organoleptic properties of a composition according to the invention, the composition may include one or more flavouring agents, such as, e.g., menthol flavour, eucalyptus, mint flavour and/or L-menthol, normally present (total concentration of flavouring agents) in a concentration of from about 0.5% w/w to about 12% w/w, from about 1% w/w to about 10% w/w, from about 1.5% w/w to about 9% w/w or from about 2% w/w to about 8% w/w.

Other Pharmaceutically Acceptable Excipients

As mentioned above, a composition according to the invention may further comprise a pharmaceutically acceptable excipient such as, e.g. a filler, a binder, a lubricant, a buffering agent, a stabilizing agent, a pH adjusting agent, a preservative, a coloring agent, a flavoring agent, a taste-masking agent, a sweetener etc.

The excipients are selected from the group of excipients normally used within the pharmaceutical industry for the preparation of tablets, i.e. excipients like fillers, disintegrants, binders, lubricants etc. To this end, excipients that enable direct compression are preferred. Guidance may be found in Handbook of Pharmaceutical Excipients edited by Rowe, R. C. et al., 4^{th} edition, Pharmaceutical Press, London 2003, which is hereby incorporated by reference.

Suitable fillers include celluloses and cellulose derivatives including microcrystalline cellulose, hydroxypropylcellulose, sodium carboxymethylcellulose etc.; lactose, starches including potato starch, maize starch etc.

Suitable lubricants include stearates including magnesium stearate, talc, colloidal silica dioxide etc.

As mentioned above, a suitable buffering agent may be included such as hydrogen carbonate including alkali metal hydrogen carbonates, or a carbonate including alkaline earth metal carbonates. The buffering agent may be coated before using it in a composition of the invention e.g. in order to protect nicotine from a high pH in the composition. A high pH has a negative effect on the stability of nicotine. In Example 3 herein, the buffering agent is coated with Eudragit® before admixing it with the other ingredients.

If present, sugar alcohols such as, e.g., sorbitol and/or isomalt, may be used in an concentration from about 5% w/w to about 35% w/w, such as, e.g., from about 10% w/w to about 35% w/w, from about 15% w/w to about 35% w/w or from about 20% w/w to about 30% w/w.

As mentioned above, a lozenge composition according to the invention may further comprise one or more anti-adhesives, lubricants, and/or glidants.

In specific embodiments, the one or more anti-adhesives, lubricants and/or glidants are selected from the group consisting of talc, stearates and salts thereof including magnesium stearate; and silica, and mixtures thereof.

In a specific embodiment, talc is present in a concentration from about 0.5% w/w to about 10% w/w, such as, e.g., from about 1% w/w to about 8% w/w, from about 1.25% w/w to about 6% w/w or from about 1.5% w/w to about 4% w/w, and/or magnesium stearate is present in a concentration from about 0.1% w/w to about 5% w/w, such as, e.g., from about 0.2% w/w to about 4% w/w, from about 0.3% w/w to about 3.5% w/w or from about 0.5% w/w to about 3% w/w, and/or silica is present in a concentration from about 0.1% w/w to about 4% w/w, such as, e.g., from about 0.2% w/w to about 3% w/w, from about 0.3% w/w to about 2% w/w or from about 0.4% w/w to about 1.5% w/w.

All details described above that are relevant for lozenge compositions apply mutatis mutandis to other aspects of the invention. To this end, a lozenge composition may comprise one or more pharmaceutically acceptable excipients like those mentioned above under chewing gum composition (apart from gum bases), and additive like e.g. buffering agents such as a hydrogen carbonate and anti-oxidants. If present, the concentration of the one or more antioxidant in a lozenge composition is from about 0.025% w/w to about 0.15% w/w, such as, e.g., from about 0.075% w/w to about 0.1% w/w.

In specific embodiments the invention relates to:

A nicotine-containing lozenge comprising

i) a carrier;

ii) nicotine, or a pharmaceutically acceptable salt, solvate, complex or derivative thereof,

wherein the nicotine-containing gum releases at least 7.5% w/w nicotine of the total composition within the first two minutes in the in vitro assay described in Ph.Eur or USP (paddle) using phosphate buffer pH 7.4 as dissolution medium 50 or 100 rpm in this method.

A nicotine-containing lozenge comprising

i) a carrier;

ii) nicotine, or a pharmaceutically acceptable salt, solvate, complex or derivative thereof,

wherein the in vivo uptake by a human, as measured by the content of nicotine in the human's serum, is rapid.

A nicotine-containing lozenge comprising

i) a nicotine-cellulose combination (concentration range: 0.5 to 50% w/w)

ii) a buffering agent (concentration range: 0-10% w/w such as 2-6% w/w)

iii) one or more artificial sweeteners (concentration range: 0-2% w/w such as 0.1 to 1% w/w),

iv) one or more flavouring agents (concentration range: 0-10% w/w such as 2-8% w/w), and

v) one or more pharmaceutically acceptable excipients (e.g. fillers such as fillers with sweetening ability like sugar alcohols) (concentration range: 10-99.5% w/w such as 20-95% w/w, 30-90% w/w, 40-85% w/w or 50-80% w/w).

A method of delivering nicotine to an individual comprising the steps of delivering to an individual the nicotine-containing lozenge as described herein.

A method for making a nicotine-containing lozenge comprising the steps of:

i) preparing a nicotine-containing composition comprising a carrier and nicotine, or a pharmaceutically acceptable salt, solvate, complex or derivative thereof, wherein the in vivo uptake by a human, as measured by the content of nicotine in the human's serum, is rapid,

ii) compressing the nicotine-containing composition optionally together with one or more pharmaceutically acceptable excipient to lozenges.

A lozenge composition comprising a nicotine-cellulose combination that has a bioavailability that is improved compared with that of Nicorette® and the improvement expressed as the relative bioavailability calculated by AUC_{0-infinity} (tested composition)/AUC_{0-infinity} (Nicorette®)×100% is at least 120% such as, e.g., at least about 130%, at least about 140% or at least about 150%—provided that the composition and Nicorette® contains the same amount of nicotine calculated as free base.

All particulars and details mentioned above relating to the lozenge aspect in general apply mutatis mutandis to the above mentioned specific embodiments.

Other Aspects

The invention also relates to a method for the preparation of a composition according to the invention. Specific details can be founds in the examples herein and a person skilled in the art will know how to find guidance e.g. from pharmaceutical handbook of how to select suitable excipient and how to prepare such compositions.

In further aspects, the invention relates to the use of a composition according to the invention as a tobacco substitute or for the alleviation of nicotine withdrawal symptoms.

In another aspect the invention, the compositions of the invention is for pharmaceutical use.

The invention is described in more details in the following figures and non-limiting examples.

Methods

In Vitro Dissolution Test

The compositions according to the invention must fulfill specific requirements with respect to in vitro release of nicotine. A suitable in vitro test depends on the specific composition in question. In general, a person skilled in the art will find guidance as to how to choose a relevant dissolution test for a specific composition in the official monographs such as, e.g., the European Pharmacopoeia. Below are described suitable dissolution tests for lozenges compositions.

The method and apparatus used were according to Ph. Eur. (paddle method).

The following examples are included to demonstrate preferred embodiments of the invention. It should be appreciated by those of skill in the art that the techniques disclosed in the examples that follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Examples

### Example 1

Preparation of a Nicotine-Cellulose Combination

Nicotine was sorbed onto microcrystalline cellulose (MCC) as described in WO 2004/056363. Accordingly, in the present example 2.40 ml nicotine was dissolved in 25 ml ethanol (99.5%). 47.6 g MCC of type PH-102 was loaded into a high-speed mixer and the nicotine was slowly added. After vacuum drying of the obtained wetted mass a fine-grained, white powder of the nicotine-cellulose combination was obtained.

### Example 10

Lozenge Compositions K, L, M and N

Nicotine-microcrystalline carrier complex made as described in Example 1 and mixed in a Turbula mixer for 10 min with the remaining ingredients (except magnesium stearate) stated in the table below. Then magnesium stearate was added to 0.5% w/w and mixing was continued for additional 3 min. The batch size was 250 g and the intended tablet weight was 625 mg.

| | Composition | | Composition | | Composition | | Composition | |
|---|---|---|---|---|---|---|---|---|
| Ingredient | K | | L | | M | | N | |
| Nicotine-MCC | 20.36 | g | 28.50 | g | 20.37 | g | 28.51 | g |
| carrier complex | | | | | | | | |
| (MCC (PH-105) | | | | | | | | |
| with 5% nicotine | | | | | | | | |
| incorporated) | | | | | | | | |
| Isomalt (DC | 206.20 | g | 198.06 | g | 193.69 | g | 185.55 | g |
| 100) | | | | | | | | |
| Carbopol (974) | — | | — | | 12.50 | g | 12.50 | g |
| Pepparmint, dry | 7.50 | g | 7.50 | g | 7.50 | g | 7.50 | g |
| flavour | | | | | | | | |
| (501432TP055) | | | | | | | | |
| Eucalyptus, dry | 7.50 | g | 7.50 | g | 7.50 | g | 7.50 | g |
| flavour | | | | | | | | |
| (501034TP0551) | | | | | | | | |
| Na₂CO₃ | 6.00 | g | 6.00 | g | 6.00 | g | 6.00 | g |
| Aspartame (fine | 1.20 | g | 1.20 | g | 1.20 | g | 1.20 | g |
| granular) | | | | | | | | |
| Magnesium | 1.24 | g | 1.24 | g | 1.24 | g | 1.24 | g |
| stearate | | | | | | | | |

The tablets were prepared from the obtained powder mixture in a single punch press (Diaf TM20) equipped with 12 mm diameter, convex punches. The excenter setting was used as an indicator of the employed compaction force as the tabletting machine was not equipped with force transducers. For the lozenge compositions K and L, which do not contain carbomer, an excenter setting of 6.1 (composition K) and 6.15 (composition L) was employed. For the lozenge compositions M and N, which do contain carbomer, an excenter setting of 7.0 was employed.

The properties of the obtained tablets were as stated in the following table:

| | Lozenge | Lozenge | Lozenge | Lozenge |
|---|---|---|---|---|
| | composition | composition | composition | composition |
| Property | K | L | M | N |
| Height, mm (10 | 6.50 | 6.60 | 5.90 | 5.90 |
| tablets) | | | | |
| Weight, mg (20 | 620 | 621 | 634 | 625 |
| tablets) | | | | |
| Disintegration | 4.90 (in vitro) | 4.90 (in vitro) | 12.5 (in vivo) | 13.8 (in vivo) |
| time, min. (6 | 4.3 (in vivo) | 5.7 (in vivo) | | |
| tablets) | | | | |
| Crushing | 6.70 | 7.30 | >20 | >20 |
| strength, kp (10) | | | | |
| Actual nicotine | 2.26 | 3.27 | 2.34 | 3.22 |
| concentration, | | | | |
| mg (10 tablets) | | | | |
| Intended | 2.5 | 3.5 | 2.5 | 3.5 |
| nicotine | | | | |
| concentration, | | | | |
| mg | | | | |

It follows from the above table, that carbopol increase the crushing strength and the disintegration time of the tablets.

The nicotine concentrations were measured by HPLC.

### Example 3

Stability of a Lozenge Composition Containing 2.5 mg of Nicotine in a Nicotine-Cellulose Combination

The drug product is a white, circular (diameter 12 mm), convex tablet containing 2.5 mg nicotine per unit with an approximate total weight of 625 mg.

The lozenge contains nitotine in the form of a nicotine-cellulose combination. In this example nicotine bitartrate has been employed. The procedure for loading the nicotine bitartrate into the cellulose is as described in Example 1 using nicotine bitartrate instead of nicotine free base.

TABLE 1
Complete composition of 2.5 mg nicotine lozenge.

| | Quantity | | |
|---|---|---|---|
| Ingredient | (mg/unit) | Function | Standard |
| Active substance | | | |
| Nicotine bitartrate dihydrate | 8.32¹ | Drug substance | Internal, Siegfried |
| Excipients | | | |
| Isomalt | 484 | Filler, sweetener | Ph. Eur. |
| Cellulose, microcrystalline, PH-105 | 44.18 | Nicotine carrier | Ph. Eur. |
| Peppermint flavour | 18.75 | Flavour | Internal, Firmenich |
| Eucalyptus flavour | 18.75 | Flavour | Internal, Firmenich |
| Sodium carbonate (Na₂CO₃) | 30.0 | Buffering agent | Ph. Eur. |
| Carbomer, Carbopol 974 | 12.5 | Swelling agent | Ph. Eur. |
| Magnesium stearate | 3.125 | Lubricant | Ph. Eur. |
| Aspartame | 3.00 | Sweetener | Ph. Eur. |
| Methacrylic acid-methyl methacrylate | 2.10 | Sodium carbonate | Ph. Eur. |
| copolymer (1:1), Eudragit L100 | | coater | |
| Ethanol, anhydrous | (32.3 + 11.9)² | Solvent | Ph. Eur. |
| Purified water | 11.9² | Solvent | Ph. Eur. |
| Total weight | 625 | | |

¹10% overage to compensate for losses during the manufacturing process. 8.32 mg nicotine bitartrate dihydrate corresponds to 2.75 mg nicotine, i.e. a 10% overage to 2.5 mg nicotine.
²Evaporates during the manufacturing process. Of ethanol 32.3 mg is used for co-solving the nicotine salt in water and 11.9 mg for dissolving Eudragit L100.

The lozenges are packed in double plastic bags of polyethylene. Final presentations are
aluminium bags, made of Transofoil® LL-OPET/polyethylene; Polyester 12 μm/Aluminium 9 μm/Polyethylene 60 μm,
   each containing 20 lozenges,

and
aluminium blisters, made of PVC/PVDC-foil 250 μm /40 g/m²−20 μm standard aluminium-foil (incl. protective lacquer layer and heat seal lacquer), each containing 10 lozenges.

The lozenge composition was subjected to an accelerated stability study. the following results were obtained:

Stability data of Zonnic™ 2.5 mg nicotine lozenge stored in aluminium bags at 40° C./75% RH, 200 units' batch No. 90905-0603-26.

| Storage | | | Related Substances (m/m %) | | |
|---|---|---|---|---|---|
| (months/ | | Assay of | | Nicotine- | Nicotine- |
| ° C./ | | Nicotine | | cis- | trans-N- |
| % RH) | Appearance | (mg/unit) | Cotinine | N-oxide | oxide |
| Limits: | White to off- | 2.37-2.63 | ≦0.5 | ≦0.5 | ≦0.5 |
| | white convex | | | | |
| | tablet | | | | |
| 0 | Conforms | 2.53 | 0.1 | n.d. | 0.1 |
| 1/40/75 | Conforms | 2.58 | 0.1 | <0.05 | <0.1 |
| 2/40/75 | Conforms | 2.63 | 0.1 | 0.1 | n.d. |
| 3/30/65 | Conforms | 2.55 | <0.1 | <0.1 | <0.05 |
| 6/30/65 | Conforms | 2.52 | <0.05 | 0.1 | <0.1 |

n.d. = not detected

Moreover, the composition was tested in a consumer test including ten subjects. A lozenge composition according to the invention was compared with a commercially available product “Commit”. 2.5/5 indicates a composition according to the invention comprising 2.5 mg of nicotine and having a disintegration time of about 5 minutes. 3.5/15 indicates a composition according to the invention comprising 3.5 mg of nicotine and having a disintegration time of about. For “Time to effect” the following comments were given:

| | 2.5/5 | Commit | 3.5/15 |
|---|---|---|---|
| Too long | 3 | 6 | 6 |
| OK | 7 | 2 | 2 |
| Too fast | 0 | 2 | 0 |

The results show that 15 minutes disintegration time leads to a product with a too long “time to effect”, whereas the product with a 5 minutes disintegration time was acceptable.

References

All patents and publications mentioned in the specification are indicative of the levels of those skilled in the art to which the invention pertains. All patents and publications are herein incorporated by reference to the same extent as if each individual publication was specifically and individually indicated to be incorporated by reference.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

1-42. (canceled)

43. A lozenge composition for achievement of a fast onset of action of nicotine after application of the solid dosage form to the oral cavity of a subject, comprising a nicotine-cellulose combination and one or more pharmaceutically acceptable excipients, wherein
i) the nicotine-cellulose combination is a solid material composed of a cellulose, which has sorbed an amount of nicotine in voids or pores within the cellulose,
ii) the concentration of nicotine in the nicotine-cellulose combination is from 3% to 20% w/w,
iii) the concentration of the nicotine-cellulose combination in the composition is from 2% to 20% w/w,
iv) the nicotine content in the composition is from 0.5 mg to 10 mg, and
v) the composition—when subjected to an in vitro dissolution test using method and apparatus in accordance with Ph. Eur. (paddle method)—within the first 2 minutes after start of the test releases nicotine with a release rate corresponding to 7.5% w/w or more of the total content in the composition per minute.

44. The composition of claim 43 wherein the composition has a shelf-life of 24 months or more when stored protected from light at a temperature not exceeding 25° C.

45. The composition of claim 43 wherein said release rate within the first 2 minutes after start of the test is 10% w/w or more of the total content in the composition per minute.

46. The composition of claim 43 wherein said release rate within the first 2 minutes after start of the test is 17% w/w or more of the total content of nicotine in the composition per minute.

47. The composition of claim 43 wherein at least 65% w/w of the total content of nicotine in the composition is released within 5 minutes when subjecting the composition to said in vitro dissolution test.

48. The composition of claim 43 wherein at least 75% w/w of the total content of nicotine in the composition is released within 10 minutes when subjecting the composition to said in vitro dissolution test.

49. The composition of claim 43 wherein said voids and/or pores at least partially comprise said nicotine.

50. The composition of claim 43 wherein the cellulose is a cellulose derived from a plant, an algae, a bacterium, a fungi, or combinations thereof

51. The composition of claim 43 wherein the cellulose has a surface area of at least 0.7 m²/g.

52. The composition of claim 43 wherein the cellulose is a crystalline cellulose including a microcrystalline cellulose.

53. The composition of claim 43 wherein the cellulose is a microcrystalline cellulose, which is selected from the group consisting of AVICEL® grades PH-100, PH-102, PH-103, PH-105, PH-112, PH-113, PH-200, PH-300, PH-302, VIVACEL® grades 101, 102,12, 20 and EMOCEL® grades 50M and 90M, and the like, and mixtures thereof.

54. The composition of claim 52 wherein said microcrystalline cellulose is a synthetic or semi-synthetic cellulose, or it is derived from a natural cellulose.

55. The composition of claim 43 wherein the mean particle size of the cellulose is in a range of from about 15 to about 250 μm.

55. The composition of claim 43 wherein the concentration of the nicotine-cellulose combination in the composition is from about 4% w/w to about 19% w/w.

56. The composition of claim 43 wherein the concentration of nicotine in the composition is in a range from about 0.1% w/w to about 15% w/w as calculated as free nicotine base.

57. The composition of claim 43 wherein the composition has a nicotine content of from about 1 mg to about 8 mg as calculated as free nicotine base.

58. The composition of claim 43 wherein the composition comprises 1.25 mg of nicotine calculated as free nicotine base.

59. The composition of claim 43 wherein the composition comprises 2.5 mg of nicotine calculated as free nicotine base.

60. The composition of claim 43 wherein the composition comprises 5 mg of nicotine calculated as free nicotine base.

61. The composition of claim 43 wherein the nicotine is selected from the group consisting of nicotine base, nicotine hydrochloride, nicotine dihydrochloride, nicotine monotartrate, nicotine bitartrate, nicotine sulfate, nicotine zinc chloride such as nicotine zinc chloride monohydrate and nicotine salicylate.

62. The composition of claim 43 wherein nicotine in the nicotine-cellulose combination is in its free base form.

63. The composition of claim 43 wherein the composition further comprises one or more buffering agents.

64. The composition of claim 63 wherein the one or more buffering agents is selected from the group consisting of acetates, glycinates, phosphates, glycerophosphates, citrates such as citrates of alkaline metals, carbonates, hydrogen carbonates, and borates, and mixtures thereof.

65. The composition of claim 63 wherein the one or more buffering agents are present in a concentration from about 0.1% w/w to about 10% w/w.

66. The composition of claim 63 wherein the one or more buffering agents is coated.

67. The composition of claim 63 wherein the composition further comprises one or more sweeteners.

68. The composition of claim 67 wherein the concentration of the one or more sweeteners is at least about 0.05% w/w.

69. The composition of claim 43 wherein the composition further comprises one or more anti-oxidants.

70. The composition of claim 69 wherein the one or more anti-oxidants are present in a concentration of from about 0.05% w/w to about 0.3% w/w.

71. The composition of claim 69 wherein the concentration of the one or more anti-oxidants in the composition is of from about 0.025% w/w to about 0.15% w/w.

72. The composition of claim 43 wherein the composition comprises one or more flavouring agents.

73. The composition of claim 72 wherein the total concentration of flavouring agents in the chewing gum composition is from about 0.5% w/w to about 12% w/w.

74. The composition of claim 72 wherein the concentration of nicotine in the nicotine-cellulose combination is from about 4% w/w to about 19% w/w.

75. The composition of claim 43 wherein the concentration of nicotine or a pharmaceutically acceptable salt, solvate or complex in the nicotine-cellulose combination is at the most about 15% w/w and the concentration being calculated as the nicotine base.

76. The composition of claim 43 wherein the composition comprises a pharmaceutically acceptable excipient.

77. The composition of claim 76 wherein the the composition comprises one or more anti-adhesives, lubricants and/or glidants selected from the group consisting of talc, stearates and salts thereof including magnesium stearate; and silica, and mixtures thereof.

78. The composition of claim 76 wherein the composition comprises talc in a concentration from about 0.5% w/w to about 10% w/w.

79. The composition of claim 76 wherein the composition comprises magnesium stearate in a concentration from about 0.1% w/w to about 5% w/w.

80. The composition of claim 76 wherein the composition comprises silica in a concentration from about 0.1% w/w to about 4% w/w.

81. A method for treating a subject suffering from or susceptible to nictone addiction, comprising administering to the subject a composition of claim 43.

82. A method for preparation of a lozenge composition defined in claim 43 comprising mixing a nicotine-cellulose combination with one or more pharmaceutical acceptable excipients and forming the resulting mixture it into a tablet by compression.
